(19) United States
(12) Patent Application Publication
(10) Pub. No.: US 2012/0151829 A1
(43) Pub. Date: Jun. 21, 2012

(54) PROCESS FOR CATALYTIC CRACKING OF AQUATIC MICROBIAL BIOMASS

(76) Inventors: Colin John SCHAVERIEN, Amsterdam (NL); Nicolaas Wilhelmus Joseph Way, Amsterdam (NL); Johan Willem Gosselink, Amsterdam (NL)

(21) Appl. No.: 12/973,169

(22) Filed: Dec. 20, 2010

(51) Int. Cl. C10L 1/22; C12P 5/00

(52) U.S. Cl. 44/333; 435/166

(57) ABSTRACT

A process for catalytically cracking aquatic microbial biomass is provided. Aquatic microbial biomass is catalytically cracked over a cracking catalyst at a temperature of at least 450° C. to obtain a product stream. At least one hydrocarbon fraction is separated from the product stream.

### TECHNICAL FIELD OF THE INVENTION

The present invention relates to a process for catalytic cracking of aquatic microbial biomass into cracked products.

### BACKGROUND OF THE INVENTION

Various processes for catalytic cracking of heavy hydrocarbons are known in the art. In these processes, heavy hydrocarbons, such as heavy oils and vacuum residues, are brought in contact with a cracking catalyst and are converted into lighter products having lower boiling points. Exemplary descriptions of such processes have been provided in U.S. Pat. No. 4,917,790 and in U.S. Pat. No. 6,905,591.

However, with the diminishing supply of crude oil, use of renewable energy sources is becoming increasingly important for the production of chemicals and fuel products. Plant and animal biomass are sources of lipids, which can be cracked to produce chemicals and fuel products. One of the advantages of using biomass is that the CO₂ balance is more favorable as compared with the conventional hydrocarbon feedstock.

EP-A-1970425 describes a process for producing gaseous and liquid fuels by catalytically cracking lipids derived from high viscosity carbon-based energy carrier materials, including aquatic biomass. WO-A-2009/000838, describes a process wherein aquatic biomass containing protein, carbohydrates and lipids is used as a source for bio-oil. However, WO-A-2009/000838 and EP-A-1970425 disclose using a cracking temperature of below 450° C., and only yield products in low yields and with limited selectivity.

Kitazato et al. describe in their article titled “Catalytic cracking of hydrocarbons from microalgae”, International Chemical Engineering, Volume 31, no 3, July 1991, a process for the production of gasoline by catalytic cracking of hydrocarbons extracted from the microalgae Botryococcus braunii Berkeley (a green algae). For the catalytic cracking process a commercial FCC zeolite was used. Exemplified reaction conditions included temperatures in the range from 450 to 500° C. Also Kitazato et al. teach, however, that low temperatures of cracking (below 450° C.) are necessary to ensure a high yield of gasoline. In addition, the use of a catalyst comprising 100 wt % zeolite is too expensive for commercial operation.

The industrial scale extraction of lipids typically involves the use of a large amount of volatile and flammable organic solvent, thereby creating hazardous and expensive operating conditions. Further, the organic solvent waste stream produced requires further treatment before being released, thereby adding to the time consumed and the overall operating cost of the process.

It is therefore evident that the process for extracting lipids from aquatic microbial biomass is complex, expensive, and time consuming, and may reduce the efficiency and economics of the overall fuel production process.

It would be an advancement in the art to provide an economical and efficient process for obtaining usable chemicals and fuel products from aquatic microbial biomass.

### SUMMARY OF THE INVENTION

Accordingly, the present invention provides a process for catalytic cracking of aquatic microbial biomass comprising contacting the aquatic microbial biomass with at least one cracking catalyst at a temperature of at least 450° C. to obtain a product stream and separating at least one hydrocarbon fraction from the product stream.

According to a further embodiment, the present invention provides a hydrocarbon fraction obtainable according to the process, comprising amines, pyrroles, pyridines, quinolines, indoles, acridines and carbazol. More preferably such a hydrocarbon fraction obtainable according to the process, comprising less than 1000 ppmw sulfur.

According to another embodiment, the present invention provides a gasoline composition comprising a hydrocarbon fraction according to the invention, and one or more additives.

It was found that, when feeding aquatic microbial biomass, contrary to the teachings in the prior art, a higher cracking temperature gives a higher conversion and hence more valuable lighter products (that is, more products having a boiling point below 221° C. such as, for example, LPG and gasoline).

The process according to the invention is further advantageous because aquatic microbial biomass, such as for example microalgae, has high growth rates, utilises a large fraction of solar energy and can grow in conditions that are not favourable for terrestrial biomass. Additionally, aquatic microbial biomass, such as microalgae, consumes CO₂ at a high rate, and may reduce the carbon footprint of the overall process.

### DETAILED DESCRIPTION OF THE INVENTION

It has now been found that directly cracking aquatic microbial biomass over a cracking catalyst at a temperature of at least 450° C. results in an economical, efficient, and faster process of obtaining cracked products.

By aquatic microbial biomass is herein understood biomass comprising microbial organisms living in an aquatic environment. Aquatic microbial biomass as referred to in the disclosed process includes, for example, microalgae, macroalgae, fungi. Preferably, the aquatic microbial biomass comprises microalgae, more preferably marine microalgae.

More preferably the aquatic microbial biomass is a biomass comprising at least microalgal proteins, microalgal carbohydrates and microalgal lipids.

In one embodiment the aquatic microbial biomass may have been pretreated before being contacted with the at least one cracking catalyst, provided that the aquatic microbial biomass has essentially not undergone any extraction step wherein lipids are extracted from the aquatic microbial biomass.

Microalgae as referred to in the present invention are a large and diverse group of usually autotrophic microorganisms that can be unicellular or multicellular. The microalgae preferably have a diameter smaller than 1 mm, more preferably a diameter smaller than 0.6 mm and still more preferably a diameter smaller than 0.4 mm. The diameter is measured at its largest point. Most preferably the microalgae comprises a diameter in the range from 0.5 to 200 micrometer, even more preferably in the range from 1 to 100 micrometer. The microalgae contain lipids, for example, monoglycerides, diglycerides, and triglycerides, which are esters of glycerol and fatty acids; and phospholipids, which are esters of glycerol and phosphate group-substituted fatty acids. These lipids are long chain hydrocarbons and are converted into lower boiling hydrocarbons by the cracking process of the present invention.

The microalgae can be cultivated under difficult agro-climatic conditions, including cultivation in freshwater, saline water, moist earth, dry sand, and other open-culture conditions known in the art. The microalgae can also be cultivated and genetically engineered in controlled closed-culture systems, for example, in closed bioreactors. Preferably, the microalgae used in the present invention are marine microalgae cultivated in fresh water, saline water, or other moist conditions, and more preferably the marine microalgae are cultivated in saline water. Yet more preferably, the marine microalgae are cultivated in open-culture conditions, for example, in open ponds. These marine microalgae can include members from various divisions of algae, including, for instance, diatoms, pyrrophyta, ochrophyta, chlorophyta, euglenophyta, dinoflagellata, chrysophyta, phaeophyta, rhodophyta, and cyanobacteria. Preferably, the marine microalgae are members from the diatoms or ochrophyta division, more preferably from the raphid, araphid, and centric diatom family. Yet more preferably, the marine microalgae are Chlorella microalgae.

Preferably the marine microalgae are cultivated in a photo-bioreactor by feeding CO₂ and nutrients, followed by injection into open ponds.

The marine microalgae is then preferably harvested, partially or wholy dried, and milled. Thereafter, the partially or wholly dried and milled marine microalgae can be used as a feed for the catalytic cracking process.

In one embodiment the process according to the invention is therefore a process for catalytic cracking of marine microalgae and comprises:

a) harvesting of marine microalgae;
b) partially or wholly drying of the harvested marine microalgae obtained in step a);
c) milling of the partially or wholy dried harvested marine microalgae obtained in step b);
d) contacting the partially or wholy dried and milled marine microalgae obtained in step c) with at least one cracking catalyst at a temperature of at least 450° C. to obtain a product stream; and separating one or more hydrocarbon fractions from the product stream.

Even more preferably the process according to the invention consists of the above mentioned steps.

The process of catalytic cracking of the aquatic microbial biomass, for example the partially or wholy dried and milled marine microalgae, preferably comprises a catalytic cracking step, which may be followed by a catalyst regeneration step.

More preferably the catalytic cracking process includes a catalytic cracking step, in which the cracking reaction takes place in the presence of a catalyst; a regeneration step, in which the catalyst is regenerated, for example by burning off the coke deposited on the catalyst as a result of the reaction, to restore the catalytic activity; and a recycle step, wherein the regenerated catalyst is recycled to the catalytic cracking step. The heat generated in the exothermic regeneration step is preferably employed to provide energy for the endothermic cracking step. Further, it is preferred to include a stripping step between the cracking and the regeneration steps to remove cracking products from the catalyst by stripping before regeneration.

Still more preferably, the catalytic cracking step comprises contacting the partially or wholy dried and milled microalgae with a cracking catalyst, preferably in the reaction zone of a fluidized catalytic cracking (FCC) apparatus. The reaction temperature preferably ranges from equal to or more than 450° C. to equal to or less than 650° C., more preferably from equal to or more than 480° C. to equal to or less than 600° C., and most preferably from equal to or more than 480° C. to equal to or less than 560° C. The pressure in the reaction zone preferably ranges from equal to or more than 0.5 bar to equal to or less than 10 bar (0.05 MPa-1 MPa), and more preferably from equal to or more than 1.0 bar to equal to or less than 6 bar (0.1 MPa to 0.6 MPa). The residence time of the cracking catalyst in the reaction zone preferably ranges from 0.1 seconds to 15 seconds, more preferably from 0.5 seconds to 10 seconds. The product stream obtained from the catalytic cracking step may be separated into one or more hydrocarbon fractions using, for example, a fractionator.

According to the invention, a catalyst to feedstock mass ratio ranging from equal to or more than 3 to equal to or less than 8 is used. Preferably, the catalyst to feedstock mass ratio used is at least 3.5. The use of the higher catalyst to feedstock mass ratio results in an increase in conversion.

The process according to the invention may further preferably comprise the catalyst regeneration step comprising burning off the coke to restore the catalyst activity by combusting the cracking catalyst in the presence of an oxygen-containing gas in a regenerator. The regeneration temperature preferably ranges from equal to or more than 575° C. to equal to or less than 950° C., more preferably from equal to or more than 600° C. to equal to or less than 850° C. The pressure in the regenerator preferably ranges from equal to or more than 0.5 bar to equal to or less than 10 bar (0.05 MPa to 1 MPa), more preferably from equal to or more than 1.0 bar to equal to or less than 6 bar (0.1 MPa to 0.6 MPa).

Cracking catalysts suitable for use in the process according to the invention are well known in the art. Preferably, the cracking catalyst comprises a zeolitic component and more preferably an amorphous binder. Examples of such binder materials include silica, alumina, titania, zirconia and magnesium oxide, or combinations of two or more of them. More preferably, the catalyst used for the invention is a commercial equilibrium catalyst.

The zeolite is usually a large pore zeolite. The large pore zeolite includes a zeolite comprising a porous, crystalline aluminosilicate structure having a porous internal cell structure on which the major axis of the pores is in the range of 0.62 nanometer to 0.8 nanometer. The axes of zeolites are depicted in the ‘Atlas of Zeolite Structure Types’ of W. M. Meier, D. H. Olson, and Ch. Baerlocher, Fourth Revised Edition 1996, Elsevier, ISBN 0-444-10015-6. Examples of such large pore zeolites include FAU or faujasite, preferably synthetic faujasite, for example, zeolite Y or X, ultra-stable zeolite Y (USY), Rare Earth zeolite Y (REY), and Rare Earth USY (REUSY). According to the present invention, USY is preferably used as the large pore zeolite

The cracking catalyst can also comprise a medium pore zeolite. The medium pore zeolite that can be used according to the present invention is a zeolite comprising a porous, crystalline aluminosilicate structure having a porous internal cell structure on which the major axis of the pores is in the range of 0.45 nanometer to 0.62 nanometer. Examples of such medium pore zeolites are of the MFI structural type, for example, ZSM-5; the MTW type, for example, ZSM-12; the TON structural type, for example, theta one; and the FER structural type, for example, ferrierite. According to the present invention, ZSM-5 is preferably used as the medium pore zeolite.

According to another embodiment, a blend of large pore and medium pore zeolites may be used. The ratio of the large pore zeolite to the medium pore size zeolite in the cracking catalyst is preferably in the range of 99:1 to 70:30, more preferably in the range of 98:2 to 85:15.

The total amount of the large pore size zeolite and/or medium pore zeolite that is present in the cracking catalyst is preferably in the range of 5 wt % to 40 wt %, more preferably in the range of 10 wt % to 30 wt %, and even more preferably in the range of 10 wt % to 25 wt % relative to the total mass of the cracking catalyst, the remainder being amorphous binder.

According to the invention, the reaction zone is usually an elongated tube-like reactor, preferably a vertical reactor in which the aquatic microbial biomass and the cracking catalyst flow in an upward direction. The aquatic microbial biomass and the cracking catalyst may also flow in a downward direction. Combinations of the downward and the upward flow are also within the scope of the present invention.

According to an embodiment of the invention, the CO2 produced in industrial processes such as the catalytic cracking process of the invention may be used for cultivation and propagation of the microalgae. This process integration preferably mitigates CO2 emissions from the overall process and facilitates cultivation of microalgae.

The product stream can comprise products that may include gaseous hydrocarbons with four or less carbon atoms, gasoline, diesel, cycle oils and other hydrocarbons.

The product stream, comprising cracked hydrocarbons, obtained from the FCC apparatus is preferably separated into one or more hydrocarbon fractions using, for example, fractionation process. Preferably, these hydrocarbon fractions include gaseous hydrocarbons such as C4-C5 hydrocarbons, liquid hydrocarbons such as gasoline, light cycle oils (LCO) and heavy cycle oils (HCO). The remaining product stream includes coke, water, acids such as acetic acid, inorganic compounds, for example, NaCl, MgO, Al₂O₃, SiO₂, P₂O₅, K₂O, CaO, Fe₂O₃, MnO₂, TiO₂, and gases such as CO₂ and CO. According to an embodiment, the total product stream composition includes a gasoline fraction ranging from 2 wt % to 12 wt %, preferably from 4 wt % to 10 wt %, as measured by Gas Chromatograph Distillation (GCD) according to the methods described in ASTM D-2887. The total product stream composition also includes a C4 hydrocarbon fraction ranging from 1 wt % to 10 wt %, preferably from 2 wt % to 8 wt % of the total product stream composition (ASTM D-2887). Further, the product stream also includes a light cycle oil fraction ranging from 5 wt % to 20 wt %, preferably from 10 wt % to 15 wt % of the total product stream composition (ASTM D-2887).

These hydrocarbon fractions may undergo further processing before they are provided for commercial use. Examples of such processing steps may include desulfurization, cracking of heavier fractions, for example LCO, and addition of one or more additives.

The commercial products obtained from these hydrocarbon fractions are also within the scope of the invention. For example, the gasoline fraction may be desulfurized to reduce the sulfur content to less than 1000 ppmw, preferably to less than 500 ppmw, more preferably to less than 200 ppmw, to obtain a gasoline product. One or more additives may be added to the desulfurized gasoline product to obtain a gasoline composition for commercial use. The additives may include performance enhancers such as anti-oxidants, corrosion inhibitors, ashless detergents, dehazers, dyes, lubricity improvers, and synthetic or mineral oil carrier fluids. Examples of such suitable additives may also be identified in U.S. Pat. No. 5,855,629, which is incorporated herein by reference. For the purpose of the invention, it should be understood that the one or more additives can be added separately to the gasoline product or can be blended with one or more diluents, forming an additive concentrate, and together added to the gasoline product. The gasoline composition according to the invention usually comprises a major amount (more than 50 wt %) of the gasoline product and a minor amount of the one or more additives described above, preferably ranging from 0.005 wt % to 10 wt %, more preferably from 0.01 wt % to 5 wt %, and most preferably from 0.02 wt % to 1 wt %, based on the gasoline composition

The C4 hydrocarbon fraction obtained from the fractionation zone may also be processed to obtain a commercial fuel product. Preferably, the product is LPG. The C4 fraction may be liquefied, and desulfurized to reduce the sulfur content to less than 1000 ppmw sulfur, preferably less than 500 ppmw sulfur, and more preferably less than 200 ppmw sulfur. Thereafter, one or more additives such as anti-oxidants, corrosion inhibitors, ashless detergents, dehazers, dyes, lubricity improvers, synthetic or mineral oil carrier fluids may be added to obtain an LPG composition for commercial use.

Similarly, the light cycle gas oil fraction may also be used for the production of heavy fuel oils, may be cracked further for conversion into additional lower boiling hydrocarbons or may be used for preparation of a lubricating oil composition.

It may be understood that processing of the hydrocarbon fractions above is well known in the art and is in no way limiting to the scope of the invention. While some of the methods have been described herein, several other processes may be used to convert the hydrocarbon fractions into commercially usable products. These processes may include isomerization, cracking into more valuable lighter products, blending with other fuels for commercial use, and other similar uses that have been disclosed in the art.

The invention is further illustrated by the following experiments.

### Experiment 1

A batch of marine microalgae of species Chlorella, was partially dried and milled. The partially dried and milled microalgae were analysed using inductively coupled plasma atomic emission spectrometry (ICP-AES) and was found to include the following (see Table 1):

TABLE 1
Elemental content of partially dried and milled microalgae

| | |
|---|---|
| Al (ppmw) | 5201 |
| B (ppmw) | 222 |
| Ca (ppmw) | 3101 |
| Fe (ppmw) | 2510 |
| K (ppmw) | 7206 |
| Mn (ppmw) | 505 |
| Ni (ppmw) | 153 |
| Si (ppmw) | 833 |
| Sr (ppmw) | 56 |
| Ti (ppmw) | 690 |
| Zn (ppmw) | 119 |
| Mg (wt %) | 1.63 |
| Na (wt %) | 3.30 |

### Experiment 2

The partially dried and milled chlorella microalgae obtained from Experiment 1 was subjected to catalytic cracking in a small-scale fluidized catalytic cracking reactor. A commercial equilibrium catalyst comprising ultra stable zeolite Y (USY) in an amorphous alumina matrix was used as the cracking catalyst. A catalyst to feedstock mass ratio of 3 was used. The reaction temperature was kept at 500° C., and the pressure was maintained at 1.1 bar (0.11 MPa). The product stream obtained was separated in a small-scale fractionator and analyzed online using gas chromatography (GC) and inductively coupled plasma mass spectrometry (ICP-MS). The results of the experiment with regard to product distribution are represented in Table 2.

TABLE 2
Product distribution

| S. | | | Yield | Yield |
|---|---|---|---|---|
| No. | Product | | (Wt %) | (Wt %) |
| 1 | Liquid | Gasoline | 5.1 | 20.2 |
| | hydrocarbon | Light cycle oil | 13.5 | |
| | fractions | Heavy cycle oil | 1.4 | |
| 2 | Water fraction | Acetic acid | 1.7 | 20.0 |
| | | Water | 18.2 | |
| | | Other acids | 0.2 | |
| 3 | Gas | CO₂ | 7 | 13.8 |
| | | CO | 0.7 | |
| | | Hydrocarbon | 3 | |
| | | fraction lighter | | |
| | | than up to and | | |
| | | including C4 | | |
| | | (C4− fraction) | | |
| | | Hydrocarbon | 3 | |
| | | fraction heavier | | |
| | | than and | | |
| | | including C5 | | |
| | | (C5+ fraction) | | |
| 4 | Coke | Carbon | 15.1 | 19.0 |
| | | Sulfur | 1 | |
| | | Nitrogen | 1.8 | |
| | | Hydrogen | 1 | |
| 5 | Inorganic | NaCl | 10 | 26.2 |
| | compounds | MgO | 6.8 | |
| | (deposited on | P₂O₅ | 2.4 | |
| | the catalyst) | SiO₂ | 2.1 | |
| | | Al₂O₃ | 1.3 | |
| | | K2O | 1.0 | |
| | | CaO + Fe₂O₃ + | 1.0 | |
| | | TiO₂ + MnO₂ | | |

| | |
|---|---|
| Total Yield % | 99.2 |

Gasoline as referred to in the invention is defined as the fraction starting with C5 isomers and boiling up to 221° C. (EP). Light Cycle Oil (LCO) as referred to in the invention is the fraction boiling from 221° C. to 370° C. (IBP-EP). Heavy Cycle Oil as referred to in the invention is the fraction boiling from 370° C. to 425° C. (IBP-EP), using the total boiling point method.

It is evident from the results represented above that the cracking of dried and milled marine microalgae results in a 99 percent mass balance and produces a desirable product stream comprising fractions that can be processed to form useful chemicals and fuel products.

The mass balance of this experiment was 99 wt %, e.g. 3.333 g marine algae powder versus 1.349 g liquid products, 0.461 g gaseous products, 0.631 g condensed cracked algae components (“coke”) and 0.873 g inorganic material deposited on the FCC catalyst surface.

The liquid organic product contained 4000 ppm of nitrogen. The amount of nitrogen containing hydrocarbons can be up to 10% depending the number of nitrogen atoms per molecule and average molecular weight of the nitrogen containing hydrocarbons.

The composition of nitrogen containing hydrocarbons was analyzed with 2D-GC with typical component groups as to be composed of amines, pyrroles, pyridines, quinolines, indoles, acridines and carbazoles. The nitrogen containing hydrocarbons included nitrogen-containing heterocyclics such as 1-methyindole and 9-methylcarbazole. The 2D-GC data was also used to calculate the concentration of gasoline (25 wt %), light cycle oil (67 wt %), heavy cycle oil (6.7 wt %) and slurry oil (1.3 wt %) fractions in the organic liquid product.

The presence of the nitrogen-containing compounds has several benefits, and makes the liquid fractions desirable fuel blending components. It is known, for instance from U.S. Pat. No. 3,922,227, that the presence of such compounds is beneficial for the oxidative stability of hydrocarbon fractions. However, in the prior art his required the use of costly additives, which need to be blended into the hydrocarbon fraction in a separate step. Moreover, the polarity if the hydrocarbon fraction is increased, making it a better blending component, for instance if Ethanol or other biofuel components need to be added to the fuel.

What is claimed is:

1. A process for catalytic cracking of aquatic microbial biomass comprising contacting the aquatic microbial biomass with at least one cracking catalyst at a temperature of at least 450° C. to obtain a product stream; and separating one or more hydrocarbon fractions from the product stream.

2. The process of claim 1 wherein the aquatic microbial biomass comprises marine microalgae preferably the diatomic microalgae.

3. A process of claim 2 for catalytic cracking of marine microalgae comprising,
a) harvesting of marine microalgae;
b) partially or wholly drying of the harvested marine microalgae;
c) milling of the partially or wholy dried harvested marine microalgae;
d) contacting the partially or wholly dried and milled marine microalgae with at least one cracking catalyst at a temperature of at least 450° C. to obtain a product stream; and separating one or more hydrocarbon fractions from the product stream.

4. The process of claim 1 wherein the cracking catalyst comprises a zeolite.

5. The process of claim 1 wherein the cracking catalyst is an ultra-stable zeolite Y (USY) catalyst.

6. The process of claim 1 wherein the ratio of the cracking catalyst to the aquatic microbial biomass is at least 3.

7. The process of claim 3 wherein the marine microalgae is a diatomic microalgae.

8. The process of claim 1 wherein the temperature ranges from 450° C. to 750° C.

9. The process of claim 8 wherein the temperature ranges from 480 to 560° C.

10. The process of claim 1 further comprising desulfurising at least one of the hydrocarbon fractions.

11. A hydrocarbon fraction produced by the process of claim 1 comprising amines, pyrroles, pyridines, quinolines, indoles, acridines and carbazol.

12. A hydrocarbon fraction produced by the process of claim 3 comprising amines, pyrroles, pyridines, quinolines, indoles, acridines and carbazol.

13. A hydrocarbon fraction of claim 11 comprising less than 1000 ppmw sulfur.

14. A hydrocarbon fraction of claim 12 comprising less than 1000 ppmw sulfur.

15. A gasoline composition comprising a hydrocarbon fraction of claim 11 and at least one or more additives.

16. A gasoline composition comprising a hydrocarbon fraction of claim 12 and at least one or more additives.
